(19) United States
(12) Patent Application Publication
(10) Pub. No.: US 2024/0055089 A1
(43) Pub. Date: Feb. 15, 2024

(54) HEALTH MANAGEMENT SYSTEM, HEALTH MANAGEMENT METHOD, AND PROGRAM

(71) Applicant: TOYOTA JIDOSHA KABUSHIKI KAISHA, Aichi-ken (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Bing XUE, Edogawa-ku (JP); Yuta MORI, Urayasu-shi (JP); Takashi HOMMA, Koto-ku (JP)

(21) Appl. No.: 18/363,739

(22) Filed: Aug. 2, 2023

(30) Foreign Application Priority Data
Aug. 9, 2022 (JP) 2022-126833

(51) Int. Cl. G16H 15/00; G16H 50/30; G16H 50/70; G06Q 30/0207; G06V 40/16; G06V 40/20

(52) U.S. Cl. CPC G16H 15/00; G16H 50/30; G16H 50/70; G06Q 30/0207; G06V 40/161; G06V 40/178; G06V 40/20

(57) ABSTRACT

Provided is a health management system that can enhance health awareness of persons existing in a predetermined zone and improve a health level of all the persons existing in the predetermined zone. The health management system according to the present disclosure executes: first determination processing of determining a state of movement of a passerby based on image data obtained by imaging the passerby with a camera installed in a predetermined zone; calculation processing of calculating an index indicating a group health state which is a health state of all persons existing in the predetermined zone based on a determination result in the first determination processing; and display processing of displaying the index calculated in the calculation processing.

### CROSS REFERENCE TO RELATED APPLICATIONS

This application is based upon and claims the benefit of priority from Japanese patent application No. 2022-126833, filed on Aug. 9, 2022, the disclosure of which is incorporated herein in its entirety by reference.

### BACKGROUND

The present disclosure relates to a health management system, a health management method, and a program.

Japanese Unexamined Patent Application Publication No. 2020-166441 discloses a technique of acquiring biological information of a target, specifying a position of the target in a distribution of a set of biological information of a group having the same attribute as the target, specifying biological information corresponding to the position in distribution of a set of biological information of a group having a different attribute from the target, and estimating a health state of the target by using a prediction model with the specified biological information as an input.

### SUMMARY

However, in the technique described in Japanese Unexamined Patent Application Publication No. 2020-166441, it is possible to estimate a health state of an individual who is a target or a predetermined group of people, but it is necessary to specify an individual or a predetermined group and acquire biological information of the individual or biological information of the group. Therefore, with the technique described in Japanese Unexamined Patent Application Publication No. 2020-166441, it is not possible to estimate a health state of all the persons existing (present) in a predetermined zone including, for example, people who enter and leave the predetermined zone such as a local public entity, and it is also not possible to improve the health state of all the persons existing in the predetermined zone.

The present disclosure has been made in view of the above problems, and provides a health management system, a health management method, and a program capable of enhancing health awareness of persons existing in a predetermined zone and improving a health level of all the persons existing in the predetermined zone.

A health management system according to the present disclosure includes: a first determination unit configured to determine a state of movement of a passerby based on image data obtained by imaging the passerby with a camera installed in a predetermined zone; a calculation unit configured to calculate an index indicating a group health state which is a health state of all persons existing in the predetermined zone based on a result of the determination in the first determination unit; and a display unit configured to display the index calculated by the calculation unit. In the health management system, since information indicating the health state of the entire predetermined zone for the persons existing in the predetermined zone can be displayed, it is possible to enhance health awareness of the persons existing in the predetermined zone and improve the health level of all the persons existing in the predetermined zone.

The display unit may display the index calculated for each predetermined zone in a ranking format. As a result, in the health management system, health state comparison with another predetermined zone can be made, so that it is possible to further enhance the health awareness of persons existing in the predetermined zone and further improve the health level of all the persons existing in the predetermined zone.

The display unit may include a display apparatus disposed outdoors in the predetermined zone. As a result, in the health management system, the health state of the predetermined zone can be confirmed outdoors, and thus, it is possible to change the behavior of persons outdoors from when the health state is confirmed, and it is possible to further improve the health level of all the persons existing in the predetermined zone.

The first determination unit may determine the state of movement of the passerby, and the first determination unit may determine a health state of the passerby based on face image data included in the image data. As a result, the health management system can determine a health state of a passerby in a more multidimensional manner, so that information indicating a health state of the entire predetermined zone to be displayed can be made more accurate.

The calculation unit may calculate a statistical value of passing speeds of all the persons existing in the predetermined zone based on the result of the determination in the first determination unit, and the display unit may display the statistical value as the index or as a part of the index. As a result, in the health management system, information indicating the health state of the entire predetermined zone to be displayed can be made more easily understandable for people, so that it is possible to further improve the health level of all the persons existing in the predetermined zone.

Here, the calculation unit may determine a target value of the passing speed based on the statistical value, and the display unit may display the statistical value and the target value as the index or as a part of the index. As a result, with the health management system, since persons in the predetermined zone can have a sense of goal, it is possible to further improve the health level of all the persons existing in the predetermined zone.

The health management system may further include a resident determination unit configured to determine whether or not the passerby is a resident residing in the predetermined zone based on the image data and determine an actual age of the resident in a case where the passerby is the resident, in which the calculation unit may calculate a first statistical value that is a statistical value of a difference between a health age and an actual age for all persons existing in the predetermined zone and residing in the predetermined zone based on the result of the determination in the first determination unit for the resident, calculate a non-resident health age that is a health age for all persons existing in the predetermined zone and not residing in the predetermined zone based on the result of the determination in the first determination unit for persons other than the resident, and calculate a statistical value of a difference between a health age and an actual age for all the persons existing in the predetermined zone based on the first statistical value and the non-resident health age as the index or as a part of the index. As a result, in the health management system, it is possible not only to calculate the difference between the health age and the actual age for all the persons existing in the predetermined zone by using the difference between the health age and the actual age with a certain degree of accuracy for the residents, but also to make the persons in the predetermined zone have a sense of goal based on the calculation result, so that it is possible to further improve the health level of all the persons existing in the predetermined zone.

The health management system may further include: an acquisition unit configured to acquire determination target data used for determining the health state of the passerby from a terminal apparatus carried by the passerby; and a second determination unit configured to determine the health state of the passerby based on the determination target data acquired in the acquisition unit, in which the calculation unit calculates the index based on the result of determination in the first determination unit and a result of the determination in the second determination unit. As a result, the health management system can determine a health state of a passerby in a more multidimensional manner, so that information indicating a health state of the entire predetermined zone to be displayed can be made more accurate.

The health management system may further include a generation unit configured to generate a change instruction for changing predetermined information regarding life in the predetermined zone based on the index calculated by the calculation unit for each predetermined zone. As a result, in the health management system, predetermined information regarding life in the predetermined zone can be changed, and people can act based on the changed information, so that the health level of all the persons existing in the predetermined zone can be further improved.

Here, the generation unit may generate the change instruction for each attribute of a resident residing in the predetermined zone. As a result, in the health management system, predetermined information regarding life in the predetermined zone can be changed for each resident attribute, and residents can act based on the changed information, so that the health level of all the persons existing in the predetermined zone can be further improved.

The health management system may further include an incentive calculation unit configured to calculate an incentive to be given for each predetermined zone based on the index calculated by the calculation unit. As a result, in the health management system, since the incentive calculated for each predetermined zone can be given for each predetermined zone, the health level of all the persons existing in the predetermined zone can be further improved.

A health management method according to the present disclosure includes: determining, by a computer, a state of movement of a passerby based on image data obtained by imaging the passerby with a camera installed in a predetermined zone; calculating, by the computer, an index indicating a group health state which is a health state of all persons existing in the predetermined zone based on a result of the determination; and performing, by the computer, control to display the calculated index on a display apparatus externally connected to the computer or included in the computer. In the health management method, since information indicating the health state of the entire predetermined zone for the persons existing in the predetermined zone can be displayed, it is possible to enhance health awareness of the persons existing in the predetermined zone and improve the health level of all the persons existing in the predetermined zone.

A program according to the present disclosure is a program for causing a computer to execute processing of: determining a state of movement of a passerby based on image data obtained by imaging the passerby with a camera installed in a predetermined zone; calculating an index indicating a group health state which is a health state of all persons existing in the predetermined zone based on a result of the determination; and displaying the calculated index on a display apparatus externally connected to the computer or included in the computer. In the program, since information indicating the health state of the entire predetermined zone for the persons existing in the predetermined zone can be displayed, it is possible to enhance health awareness of the persons existing in the predetermined zone and improve the health level of all the persons existing in the predetermined zone.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram illustrating a configuration example of a health management system according to an embodiment;

FIG. 2 is a flowchart for explaining an example of processing in the health management system of FIG. 1;

FIG. 3 is a block diagram illustrating a more specific configuration example of the health management system of FIG. 1;

FIG. 4 is a schematic top view illustrating an arrangement example of a camera and a display apparatus in the health management system of FIG. 3;

FIG. 5 is a schematic view illustrating an example of a display image displayed on the display apparatus in the health management system of FIG. 3;

FIG. 6 is a schematic view illustrating another example of the display image displayed on the display apparatus in the health management system of FIG. 3;

FIG. 7 is a block diagram illustrating another more specific configuration example of the health management system of FIG. 1;

FIG. 8 is a schematic view illustrating an example of a display image displayed on the display apparatus in the health management system of FIG. 7;

FIG. 9 is a block diagram illustrating a configuration example of a learning system that generates a learning model used in the health management system of FIG. 3 or 7;

FIG. 10 is a diagram illustrating an example of training data used in the learning system of FIG. 9; and

FIG. 11 is a diagram illustrating an example of a hardware configuration included in an apparatus.

### DESCRIPTION OF EMBODIMENTS

Hereinafter, the present disclosure will be described with embodiments of the disclosure, but the disclosure according to the claims is not limited to the following embodiments. In addition, not all the configurations described in the embodiments are essential as means for solving the problem. Hereinafter, embodiments will be described with reference to the drawings.

### Embodiment

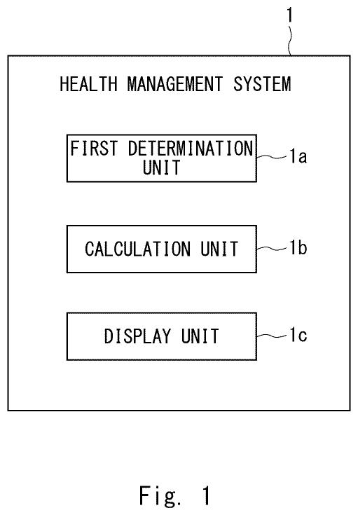

FIG. 1 is a block diagram illustrating a configuration example of a health management system according to the present embodiment. As illustrated in FIG. 1, a health management system 1 according to the present embodiment can include a first determination unit 1a, a calculation unit 1b, and a display unit 1c.

The first determination unit 1a determines a state of movement of a passerby based on image data obtained by imaging the passerby with a camera installed in a predetermined zone. The image data can be acquired as data obtained by imaging the passerby from the camera installed in the predetermined zone. In order to acquire such image data, the health management system 1 can include an image acquisition unit (not illustrated). The image acquisition unit can include a communication unit. The image acquisition unit and the camera correspond to a monitoring unit that monitors a passerby since the camera images the passerby and the image acquisition unit acquires the image data. The camera is an imaging apparatus.

Here, the predetermined zone may be an area determined in advance, and examples thereof include various geographical areas such as one local public entity, a plurality of adjacent local public entities, a commercial area such as one shopping street, and a site or a building of a company. The predetermined zone is a zone in which management of passersby is performed. Therefore, for example, even in a case where a site or a building of a company is set as the predetermined zone, passersby are not limited to employees but include visitors. In addition, for example, in a case where one of the predetermined zones is a first local public entity (for example, Tokyo Metropolis) and another one of the predetermined zones is a second local public entity (for example, Saitama Prefecture) adjacent to the first local public entity, when there is a person who visits the first local public entity even though the person resides in the second local public entity, the following situation occurs. That is, the person is determined as a passerby in the first local public entity, and is counted as a passerby existing in the first local public entity at the time of calculation described below.

In addition, as can be seen from the example in which the predetermined zone is a building of a company, an installation place of the camera is not limited to an outdoor space, and may include an indoor space. For example, the camera may be installed in one or more sensing areas arranged at a gate that is within the predetermined zone or various other places within the predetermined zone. The sensing area at each installation place can also be referred to as a sensing zone. For example, since the state of movement of the passerby can be used for health state management, the sensing zone can also be referred to as a healthcare zone. The healthcare zone can be installed as a zone in which various types of data indicating a health state are acquired when a user only passes through the zone by walking or the like. The healthcare zone can include a camera and various sensors necessary for acquiring other information, a support such as a pole or a gate that supports the camera and other sensors, and a communication unit that transmits imaging data (image data) captured by the camera and a measurement result of other sensors. As described as the installation place of the camera, the healthcare zone can be installed at least at a place where a person is likely to pass in the predetermined zone, such as an entrance of the predetermined zone, can be installed continuously, for example, at regular intervals from the place, or can be installed at a bus stop. Furthermore, as the camera is used as a security camera, security of the predetermined zone can be improved.

The state of movement of the passerby refers to a state of passage of the passerby. The movement of the passerby may refer to passage with movement, such as walking or running of a pedestrian, or traveling of a person on a bicycle. Therefore, the movement of the passerby can basically exclude movement by driving of an automobile, a motorcycle, or the like. However, for example, the state of the movement can also be determined from a face image, and thus the movement by driving of an automobile, a motorcycle, or the like can also be included in a case of making such a determination.

The calculation unit 1b calculates an index indicating a group health state which is a health state of all persons existing in the predetermined zone based on the determination result of the first determination unit 1a. The calculation unit 1b can calculate, as the index, a statistical value of a walking state, a gait, a health age, and the like, for example, by statistical computation or by using a learning model. Here, the health age generally refers to an age calculated based on various measured values obtained by performing a medical examination, but the health age here can be an age estimated from information obtained from image data in such a way as to match therewith, and the definition thereof is not limited.

In other words, the first determination unit 1a can obtain a value that enables the calculation unit 1b to calculate such an index, for example, a value such as the walking state, the gait, or the health age of each passerby, as a determination result. The calculation unit 1b can also calculate, that is, estimate, the index as an estimated value. Since the group health state refers to the health state in the predetermined zone, it can also be referred to as a zone health state. The health management system 1 performs such calculation and can thus be referred to as a health state calculation system.

The display unit 1c displays the index calculated by the calculation unit 1b. The display unit 1c can include one or a plurality of display apparatuses and a display control unit that controls display on the display apparatuses. The display unit 1c only needs to visualize the index, but can also display a result of comparison with a previous index such as comparison between the last week and this week. Furthermore, a value indicating the total health age in a predetermined period such as today, this week, or this month can be calculated and displayed as the index. In addition, similarly to a weather display method in the weather forecast, the index of each predetermined zone may be displayed on a map.

Furthermore, the first determination unit 1a, the calculation unit 1b, and the display control unit described above can be provided as a control unit that controls the entire health management system 1. The control unit can be implemented by, for example, an integrated circuit, and can be implemented by, for example, a processor such as a central processing unit (CPU), a work memory, a nonvolatile storage apparatus, and the like. A control program executed by the processor is stored in the storage apparatus, and the processor reads the program to the working memory and executes the program, so that the determination function of the first determination unit 1a, the calculation function of the calculation unit 1b, and the display control function of the display control unit can be implemented.

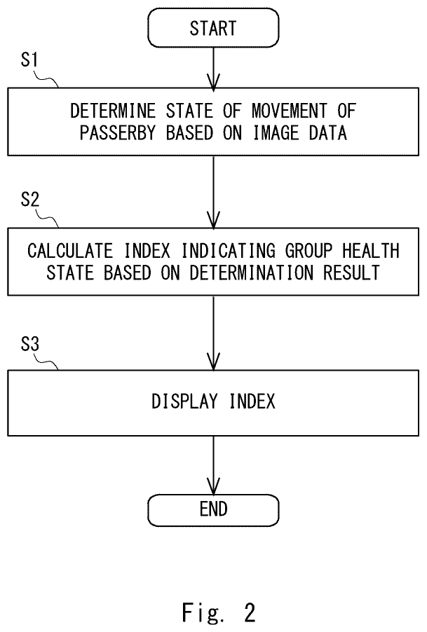

Next, an example of a health management method executed by the health management system 1 will be described with reference to FIG. 2. FIG. 2 is a flowchart for explaining an example of processing in the health management system 1 of FIG. 1.

In the health management method, first, the first determination unit 1a inputs image data obtained by imaging a passerby with the camera installed in the predetermined zone, and determines a state of movement of the passerby based on the image data (step S1). Next, the calculation unit 1b calculates an index indicating a group health state which is a health state of all persons existing in the predetermined zone based on the determination result (step S2). Next, the display unit 1c displays the calculated index (step S3), and ends the processing.

The health management method can be mainly executed by a computer, and in step S3, the computer performs control to display the calculated index on a display apparatus connected to the outside or included in the computer. The above-described control program can include a program for causing the computer to execute the processing indicated by such a health management method.

As described above, in the health management system 1, it is possible to grasp the health state of persons existing in the entire predetermined zone such as a local public entity, that is, persons existing in the predetermined zone at the time of image data acquisition, including, for example, persons who enter and leave the predetermined zone. Then, the health management system 1 can display information indicating the grasped health state of the entire predetermined zone. Therefore, in the health management system 1, it is possible to enhance health awareness of persons existing in the predetermined zone and improve the health level of all the persons existing in the predetermined zone. The health level refers to a level indicating the health state.

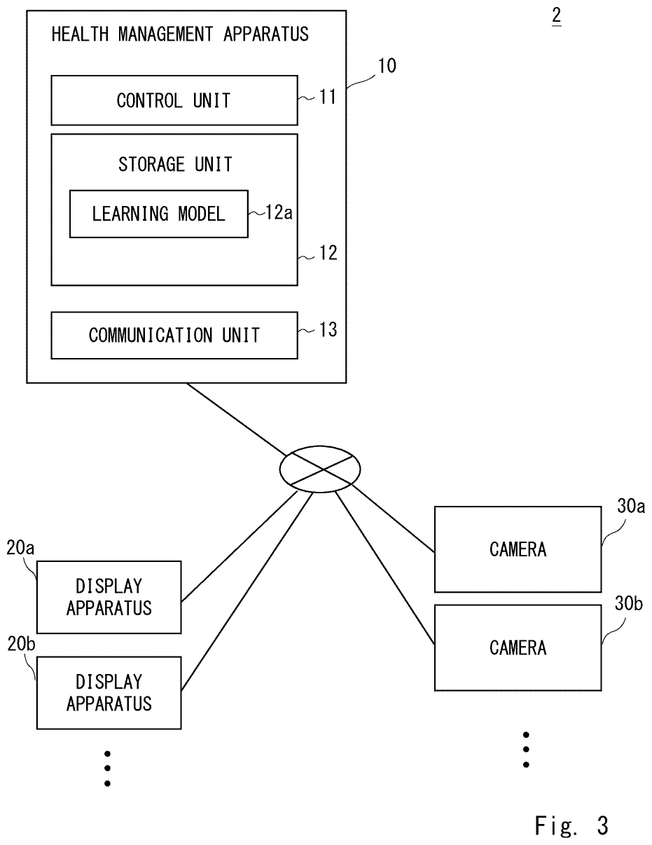

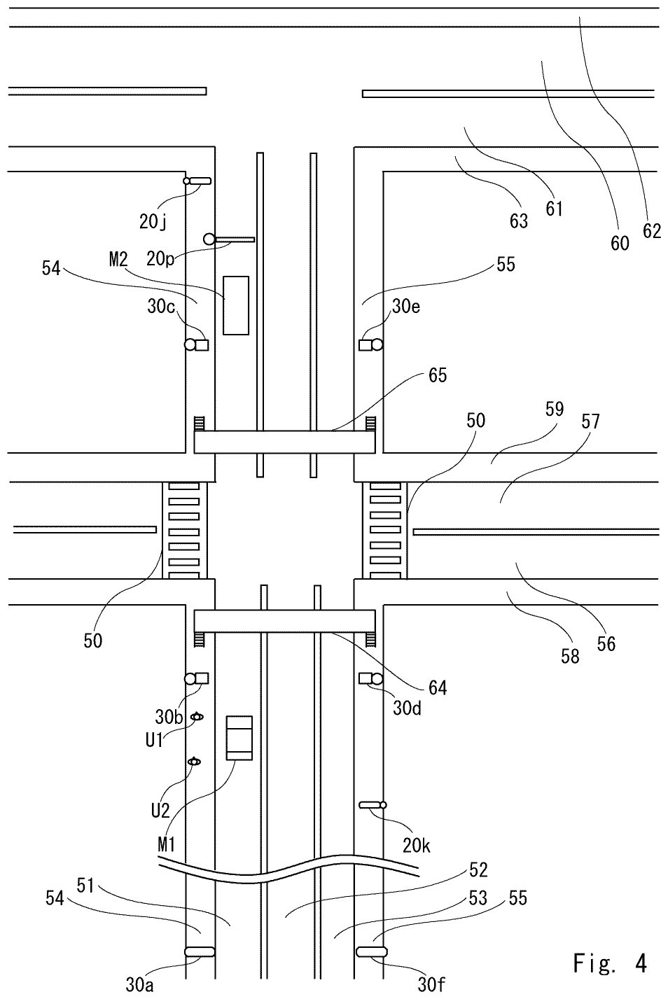

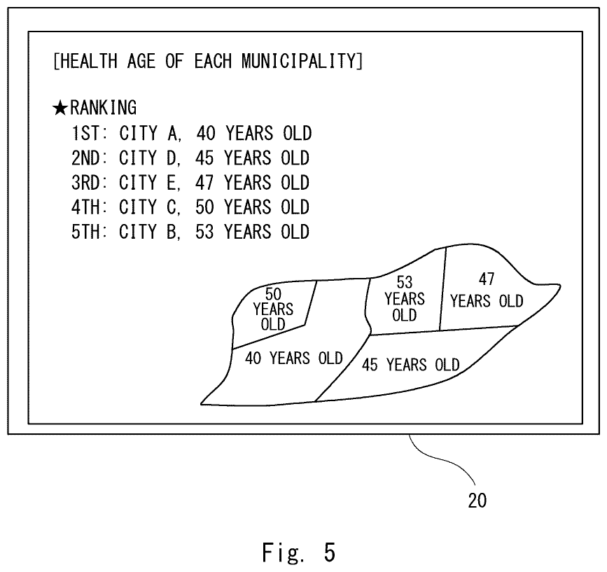

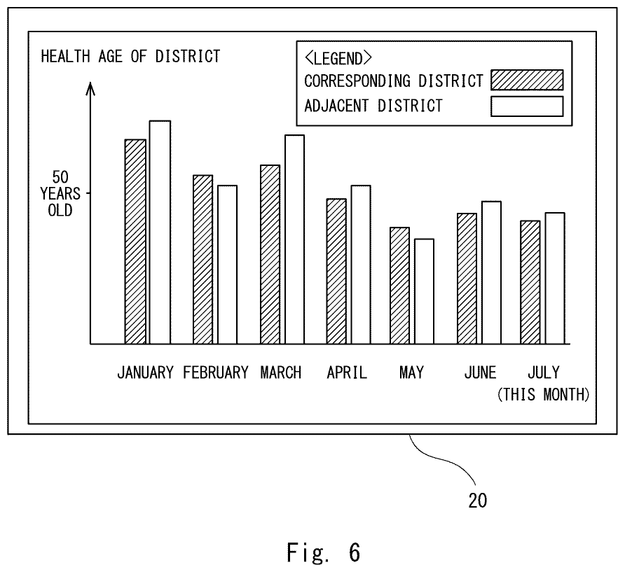

Next, a more specific configuration example of the health management system 1 will be described with reference to FIGS. 3 to 6. FIG. 3 is a block diagram illustrating a more specific configuration example of the health management system of FIG. 1. FIG. 4 is a schematic top view illustrating an arrangement example of a camera and a display apparatus in the health management system of FIG. 3. FIG. 5 is a schematic view illustrating an example of a display image displayed on the display apparatus in the health management system of FIG. 3, and FIG. 6 is a schematic view illustrating another example of the display image displayed on the display apparatus in the health management system of FIG. 3.

A health management system 2 illustrated in FIG. 3 can include a health management apparatus 10, a plurality of display apparatuses 20a, 20b, and the like, and a plurality of cameras 30a, 30b, and the like. It is a matter of course that the camera 30a and the like do not have to be included in components of an example of the health management system 1. In the following description, in a case where the display apparatuses 20a, 20b, and the like are not distinguished from one another, they are referred to as the display apparatus 20. Similarly, in a case where the cameras 30a, 30b, and the like are not distinguished from one another, they are referred to as the camera 30.

The health management apparatus 10 can include a control unit 11 that controls the entire health management apparatus 10, a storage unit 12 implemented by a storage apparatus, and a communication unit 13 implemented by a communication interface or the like that communicates with an external apparatus. The health management apparatus 10 can include, for example, a computer. The health management apparatus 10 can be configured alone, but can also be configured as a distributed system in which functions thereof are distributed.

The control unit 11 includes a first determination unit 1a and a calculation unit 1b, and has a function other than an interface for communication in a display control unit. Note that the function of the interface for communication in the display control unit is executed by the communication unit 13. The communication unit 13 also functions as an example of the image acquisition unit, and receives image data acquired by the camera 30 from the camera 30.

The control unit 11 can be implemented by, for example, a processor, a work memory, a nonvolatile storage apparatus, and the like. A control program executed by the processor is stored in the storage apparatus, and the processor reads the program to the work memory and executes the program, so that the function of the control unit 11 can be implemented. The storage unit 12 can use a partial storage region of the storage apparatus. The control program can include a program for implementing the determination function of the first determination unit 1a, a program for implementing the calculation function of the calculation unit 1b, and a program for implementing the display control function of the display control unit. In this case, the processor reads the program to the work memory and executes the program, thereby implementing the functions. Note that functions not included in the control program can be implemented by a hardware configuration.

The control unit 11 receives image data obtained by imaging (capturing an image of) a passerby with the camera 30 installed in the predetermined zone from the camera 30 via the communication unit 13, and determines a state of movement of the passerby based on the image data. The image data captured by the camera 30 and received from the camera 30 can be still image data, a series of still image data captured at predetermined intervals, or moving image data. As described above, the state of movement can be, for example, a walking state, a gait, a health age, and the like. Among them, a result of determining the walking state or the gait can be obtained as a value indicating a difference from a standard walking state or gait.

The control unit 11 calculates an index indicating a group health state which is a health state of all persons existing in the predetermined zone based on the passerby movement determination result. The control unit 11 can calculate, as the index, a statistical value of the walking state, the gait, the health age, and the like by using a learning model 12a stored in the storage unit 12. The learning model 12a is a model trained by machine learning in such a way as to receive the determination result and output the index, and an algorithm thereof or the like is not limited.

Alternatively, the control unit 11 can receive the image data obtained by imaging the passerby with the camera 30 installed in the predetermined zone from the camera 30 via the communication unit 13, and calculate the index from the image data by using a learning model stored in the storage unit 12. Unlike the learning model 12a, the learning model in this case is a model trained by machine learning in such a way as to receive the image data and output the index. An algorithm or the like of the learning model is not limited.

Then, the control unit 11 performs control to display the calculated index on the display apparatus 20. The display apparatus 20 only needs to be able to visualize the index, but may display a result of comparison with a previous index such as comparison between the last week and this week. Furthermore, a value indicating the total health age in a predetermined period such as today, this week, or this month can be calculated and displayed as the index. In addition, similarly to a weather display method in the weather forecast, the index of each predetermined zone may be displayed on a map.

A timing for such calculation and display of the index can be determined by performing processing based on image data acquired for a predetermined period such as one day, for example. In this case, the control unit 11 can perform face authentication processing on pieces of image data in order to exclude overlapping persons. Then, the control unit 11 performs processing such as selecting the oldest data, the latest data, or the like from among pieces of image data of a passerby imaged a plurality of times during a predetermined period, or taking statistics and selecting the most well-expressed image data of a state of movement of the passerby, and can use the selected image data as an input to the learning model 12a.

The storage unit 12 stores the learning model 12a described above, and also stores setting values and the like necessary for control in the control unit 11. The learning model 12a is a learning model that receives a result of determining movement of a passerby and outputs, for each predetermined zone, an index indicating a group health state which is a health state of all persons existing in the predetermined zone, and an algorithm or the like thereof is not limited. In addition, the input parameters are not limited thereto.

The learning model 12a can be obtained, for example, as follows. That is, the learning model 12a can be obtained by performing machine learning in a manner in which a data set including a result of determining movement of each passerby and ground truth data indicating a statistical value (index) obtained by performing statistical processing on the health age or the like of each passerby at this time is input to an untrained learning model. For example, a plurality of data sets in which at least one of a determination timing or the predetermined zone is different from each other can be used for training. The index of the passerby is not limited to such a statistical value, and for example, an index determined by a model creator or the like can be used. The learning model 12a can be a model that can accurately determine the index indicating the group health state by learning the data set for a number of timings or predetermined zones to the extent that over-learning does not occur. Note that a system that performs such learning will be described below.

The communication unit 13 communicates with the camera 30 via a wired network, transfers image data captured by the camera 30 to the control unit 11, communicates with the display apparatus 20 via a wired network, and outputs a display image to the display apparatus 20 under the control of the control unit 11. All the networks may be a wireless network, and a method of communication between the communication unit 13 and the camera 30 or the display apparatus 20 is not limited.

The camera 30 is a camera installed in the predetermined zone, and transmits captured image data to the health management apparatus via a wired network. A transmission timing may be, for example, a predetermined interval, or may be a timing at which a person is imaged in a case where a person detection function is provided. The camera can include a communication interface built in or be connected to a communication interface for the transmission.

Here, an example will be described in which a region illustrated in FIG. 4 is the predetermined zone, and the plurality of cameras 30 are provided in the predetermined zone. The region illustrated in FIG. 4 includes a main road including lanes (roadways) 51 to 53 and sidewalks 54 and 55, the first small and medium road including roadways 56 and 57 and sidewalks 58 and 59 and crossing the main road, and the second small and medium road including roadways 60 and 61 and sidewalks 62 and 63 and forming a T-junction with the main road at the end of the main road. In addition, in the region illustrated in FIG. 4, two crosswalks 50 that cross the roadways 56 and 57 of the first small and medium road are provided, and pedestrian bridges 64 and 65 are provided in such a way as to be able to cross the roadways 51 to 53 of the main road by walking along the first small and medium road. In addition, FIG. 4 illustrates a state in which users U1 and U2, an automobile M1, and an autonomous mobile body M2 such as an autonomous bus exist in the illustrated region. However, since persons, automobiles, or the like are always moving, only a state of a moment is illustrated here.

As the camera 30 provided in the region illustrated in FIG. 4, sensing zones 30a and 30f provided with sensors including the camera 30 are provided on the sidewalks 54 and 55 corresponding to the entrance of the predetermined zone, respectively. In addition, cameras 30b and 30c are provided on the sidewalk 54, and cameras 30d and 30e are provided on the sidewalk 55. Although not illustrated, the sensing zone can also be provided at the entrance of the region in the sidewalks 58, 59, 62, and 63. For example, the cameras 30b and 30c can be installed on poles provided on the sidewalk 54, and the cameras 30d and 30e can be installed on poles provided on the sidewalk 55. In the sensing zones 30a and 30f, the sensors and the like can be installed on an arch extending over both ends of the sidewalk 54, but the sensors and the like can also be installed on poles similarly to the camera 30b and the like.

At least one of the display apparatuses 20 provided in the health management system 2 can be a display apparatus such as a digital signage disposed outdoors in the predetermined zone. In the example of FIG. 4, a digital signage 20j provided on the sidewalk 54, a digital signage 20k provided on the sidewalk 55, and a digital signage 20p for the roadway 51 are installed as the display apparatuses 20. Since it can be said that it is desirable not to provide unnecessary information during driving, it is not necessary to display the calculated index on the digital signage 20p. By adopting such outdoor display, in the health management system 2, the health state of the predetermined zone can be confirmed outdoors, and thus, it is possible to change the behavior of persons outdoors from when the health state is confirmed, and it is possible to further improve the health level of all the persons existing in the predetermined zone.

In order to designate the display apparatus 20 as a display target, although not illustrated, the storage unit 12 stores position information indicating the position of the display apparatus 20 or information indicating a predetermined zone in which the display apparatus 20 is installed. As a result, it is possible to instruct the display apparatus 20 corresponding to the predetermined zone to display the index calculated for the predetermined zone. It is sufficient if the position information is information that specifies a position, such as information indicating latitude and longitude or information indicating coordinates on map data.

Furthermore, the control unit 11 can control the display apparatus 20 to display the index calculated for each predetermined zone in a ranking format. For example, as illustrated in FIG. 5, the display apparatus 20 can display a display image in which the calculated health age is indicated in a ranking format for each of adjacent cities A to E as the predetermined zones. In addition, FIG. 5 also illustrates an example in which the health age is displayed on a map of each city in order to facilitate understanding of a positional relationship among the cities. It is a matter of course that the name of each city can also be displayed on the map of each city. In addition, it is also possible to simultaneously display information such as an arrow indicating an increase or decrease in ranking in time series.

By adopting such display in a ranking format, in the health management system 2, health state comparison with another predetermined zone can be made, and the predetermined zones can compete with each other, so that it is possible to further enhance the health awareness of persons existing in the predetermined zone and further improve the health level of all the persons existing in the predetermined zone.

Furthermore, the control unit 11 can control the display apparatus 20 to display the index of a specific predetermined zone such as an adjacent predetermined zone and the index of a target predetermined zone in a comparable manner. For example, as illustrated in FIG. 6, the display apparatus 20 can display a display image showing a monthly change in health age calculated for a corresponding district and an adjacent district to the corresponding district.

Also by adopting such comparison type display, in the health management system 2, health state comparison with another predetermined zone can be made, and the predetermined zones can compete with each other, so that it is possible to further enhance the health awareness of persons existing in the predetermined zone and further improve the health level of all the persons existing in the predetermined zone.

As a function of the first determination unit 1a, the control unit 11 may determine a state of movement of a passerby. Further, as the function, the control unit 11 may determine a health state of the passerby based on data of a face image included in the image data. A method for determining the health state from the face image is not limited. For example, the determination can be made based on various determination criteria such as that a person whose complexion is close to the soil color is determined to be a person whose health age is old, that a person whose forehead or outer corners of the eyes have many wrinkles is determined to be a person whose health age is old, and that a person whose laugh lines are deeper is determined to be a person whose health age is old.

Furthermore, the control unit 11 can also determine a psychological state such as a stress level as a kind of the health state from data of the facial expression included in the data of the face image. Although it is assumed that there is a passerby whose face image cannot be obtained due to a problem of an angle at the time of imaging, information that is ultimately necessary is the index, and it is sufficient if statistical processing is executed. In this case, the psychological state may be determined only for a passerby whose face image has been obtained. In addition, the stress level only needs to be an index indicating the degree of stress (stress level) of the user, and the stress level can be determined as one of a plurality of predetermined levels. The degree of stress can be, for example, a degree indicating whether the user is in an excessive stress state or in a calm state. The excessive stress state can refer to a state in which a person is anxious or irritated.

As a result, the health management system 2 can determine a health state of a passerby in a more multidimensional manner, so that information indicating a health state of the entire predetermined zone to be displayed can be made more accurate.

In addition, as a function of the calculation unit 1b, the control unit 11 may calculate a statistical value of passing speeds of all the persons existing in the predetermined zone as the index or as a part of the index based on a result of determining the state of movement. For example, as the result of determining the state of movement, [a change amount of the position of the passerby specified from the image data] and [an acquisition interval of camera images corresponding to the change amount] are acquired for each passerby, and the passing speed can be calculated by [the change amount of the position of the passerby specified from the image data]/[the acquisition interval of the camera images corresponding to the change amount] for each passerby. Then, the control unit 11 can obtain the statistical value of the passing speed by performing statistical processing on the value calculated for each passerby for all the persons existing in the predetermined zone. The passing speed can also be calculated by distinguishing a walking speed of a pedestrian from a traveling speed of a bicycle. It is a matter of course that the learning model 12a can also be used to calculate the passing speed. In this case, the statistical value of the calculated passing speeds is displayed on the display apparatus 20 as the index or as a part of the index.

By visualizing and presenting the passing speed in the predetermined zone in this way, in the health management system 2, information indicating the health state of the entire predetermined zone to be displayed can be made more easily understandable for people, so that it is possible to further improve the health level of all the persons existing in the predetermined zone. That is, in the health management system 2, the average passing speed of the entire predetermined zone is increased and the health level can be improved by visualizing and presenting the passing speed in the predetermined zone.

Here, the control unit 11 may determine a target value of the passing speed based on the calculated statistical value, and cause the display apparatus 20 to display not only the statistical value but also the determined target value as the index or as a part of the index. As a result, in the health management system 2, since persons in the predetermined zone can have a sense of goal, it is possible to further improve the health level of all the persons existing in the predetermined zone.

As an index other than the passing speed, for example, a stride length and a walking speed can be calculated by recognizing a skeleton from the posture or physique of a person based on image data, a walking ability can be calculated as a part of the index based on the skeleton, stride length, and walking speed, and a statistical value of the walking ability can be calculated. The learning model can also be used for such skeleton recognition.

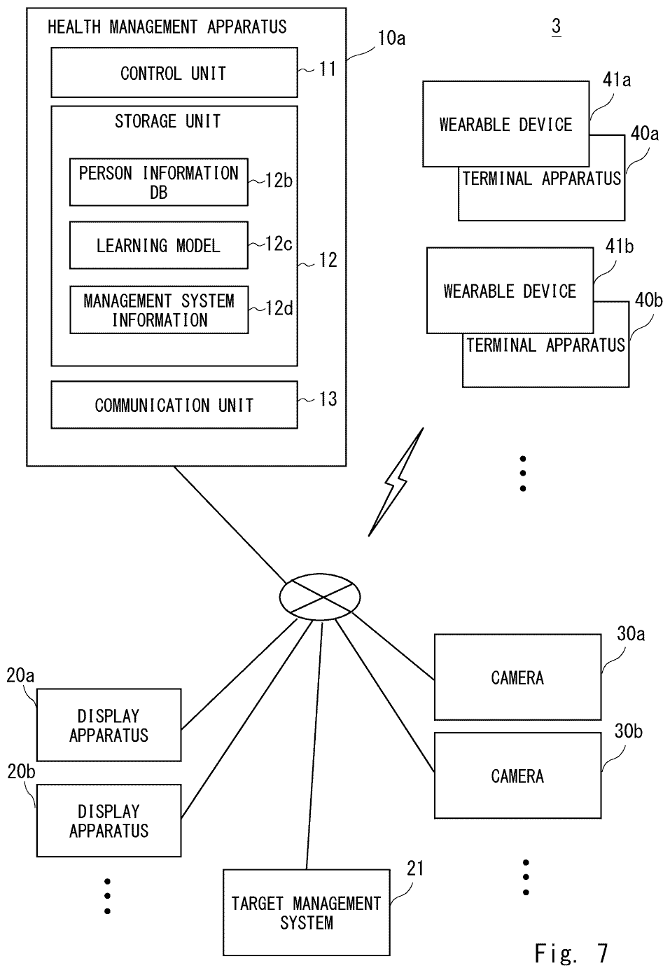

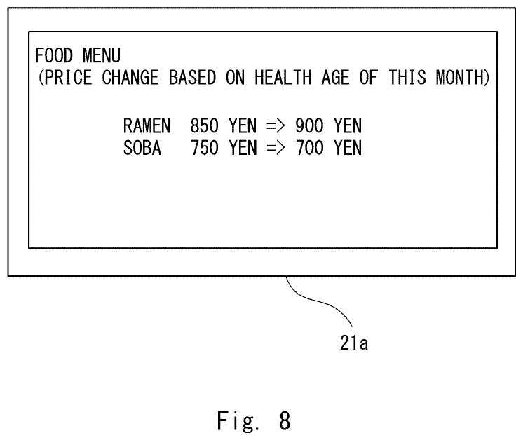

Next, another more specific configuration example of the health management system 1 will be described with reference to FIGS. 7 and 8. FIG. 7 is a block diagram illustrating another more specific configuration example of the health management system of FIG. 1. FIG. 8 is a schematic view illustrating an example of a display image displayed on a display apparatus in the health management system of FIG. 7.

A health management system 3 illustrated in FIG. 7 can include a health management apparatus 10a, a plurality of display apparatuses 20, a plurality of cameras 30, and a target management system 21. It is a matter of course that the camera 30a, the target management system 21, and the like do not have to be included in components of an example of the health management system 1. The display apparatus 20 and the camera 30 are as described in the configuration example of FIG. 3.

The health management apparatus 10a is an apparatus that controls the plurality of display apparatuses 20 and the target management system 21, and can include, for example, a computer. It can be said that the health management apparatus 10a includes a target management system control apparatus for controlling the target management system 21. The health management apparatus 10a can be configured alone, but can also be configured as a distributed system in which functions thereof are distributed. Here, an example in which one target management system 21 is included in the health management system 3 is described, but the number of target management systems to be controlled is not limited.

The target management system 21 can be a system that manages targets serving as various incentives, such as a point management system that manages points that can be used in at least the predetermined zone and can be used in product purchase services. The type of the product purchase service, the value of the given point, and the like are not limited. It is a matter of course that, for convenience, one target management system 21 will be described, but each target serving as an incentive can be constructed as a separate system.

In addition, the health management system 3 can include a sensor group for obtaining information used for control in the health management apparatus 10a as a component of an example of the health management system 1. As illustrated in FIG. 3, the sensor group can include, for example, wearable devices 41a, 41b, and the like worn by users in addition to the plurality of cameras 30. Furthermore, the health management system 3 can also include terminal apparatuses 40a, 40b, and the like that transmit information measured by the wearable devices 41a, 41b, and the like to the health management apparatus 10a, and the terminal apparatuses 40a, 40b, and the like can also be used to display an index. It is a matter of course that the camera 30a and the like, the wearable device 41a and the like, the terminal apparatus 40a and the like do not have to be included in components of an example of the health management system 1.

Similarly, in a case where the terminal apparatuses 40a, 40b, and the like and the wearable devices 41a, 41b, and the like are not individually distinguished, they are referred to as the terminal apparatus 40 and the wearable device 41, respectively.

Although an example in which the psychological state such as a stress level of a passerby is determined based on image data acquired by the camera 30 has been described, the psychological state can also be determined using vital information measured by the wearable device 41. The wearable device 41 is an example of a measuring instrument (measurement apparatus) that measures vital information of a user in order to determine the psychological state such as a stress level and other types of health states of the user with the health management apparatus 10a. The wearable device 41 may be a smart watch, a smart ring, an IC chip embedded in a human body, or the like, but is not limited thereto.

Furthermore, the vital information measured by the wearable device 41 can be, for example, information indicating one or more of a pulse rate or heart rate, a respiration (rate), a blood pressure, a body temperature, and the like. The vital information to be measured is not limited to above information and may include, for example, information indicating electrocardiogram. Further, the vital information is information whose value changes even for the same user, for example, while the person is controlling his/her bladder, while the person is running, or while the person is walking normally.

The wearable device 41 directly or indirectly transmits the acquired vital information to the health management apparatus 10a. In other words, the wearable device 41 can include a communication interface built in or be connected to a communication interface for the transmission. The wearable device 41 can be configured to measure the stress level and transmit the stress level to the health management apparatus 10a.

In a simple example, the wearable device 41 can be configured to spontaneously transmit the vital information to the health management apparatus 10a, for example, at a predetermined interval or the like. The transmission may be direct or indirect. In this case, the health management apparatus 10a can have a function of collecting and managing the vital information as a server apparatus (not illustrated) or the like. In this case, transmission of the vital information to the health management apparatus 10a may be permitted in advance in the wearable device 41 or the terminal apparatus 40 such as a portable terminal apparatus connectable to the wearable device 41. In such a spontaneous transmission example, it is not necessary to transmit a vital information transmission request in a state where the wearable device 41 is designated from the health management apparatus 10a. The vital information directly or indirectly transmitted from the wearable device 41 can include information indicating the user, and a resident determination unit to be described below can determine whether or not the user is a resident based on the information.

Regardless of whether the transmission is direct or indirect, and whether or not the vital information transmission request is necessary, the vital information is received by the communication unit 13 of the health management apparatus 10a and transferred to the control unit 11. As in this example, the health management apparatus 10a can include an acquisition unit that acquires determination target data such as the vital information used for determining a health state of a passerby from the wearable device 41 which is an example of a terminal apparatus carried by the passerby. In FIG. 3, the communication unit 13 and the control unit 11 that controls the acquisition are examples of the acquisition unit.

An example in which the wearable device 41 indirectly transmits the vital information will be described. The wearable device 41 transmits the vital information to the terminal apparatus 40 used by the user wearing the wearable device 41 by short-range wireless communication or the like, and the terminal apparatus 40 transmits the vital information to the health management apparatus 10a. The terminal apparatus 40 can be, for example, a portable phone such as a smartphone, a tablet terminal, a mobile personal computer (PC), or the like. Further, a method for the short-range wireless communication described above and below is not limited, and various methods such as Wi-Fi (registered trademark), Bluetooth (registered trademark), Bluetooth Low Energy (registered trademark), and ZigBee (registered trademark) can be adopted.

Furthermore, the terminal apparatus 40 can also generate the determination target data to be used for determination of the health state such as the psychological state from face image data captured by, for example, an included camera, and transmit the determination target data to the health management apparatus 10a. In this case, the determination target data can be obtained without the wearable device 41. However, the health management apparatus 10a can more accurately estimate the health state of the passerby by acquiring both the face image data and the vital information as the determination target data.

Further, the determination target data can be transmitted via a communication interface with which short-range wireless communication with the wearable device 41 or the terminal apparatus 40 can be performed, the communication interface being built in or connected to the camera 30.

Then, the wearable device 41 or the terminal apparatus 40 can be configured to automatically transmit the determination target data when entering an area in which wireless communication with the communication interface can be performed. In such a configuration, it is not necessary to transmit a determination target data transmission request in a state where the wearable device 41 or the terminal apparatus 40 is designated from the health management apparatus 10a. Also in this case, transmission of the determination target data to the health management apparatus 10a may be permitted in advance in the wearable device 41 or the terminal apparatus 40.

Alternatively, the wearable device 41 or the terminal apparatus 40 can periodically transmit the determination target data together with the position information to a server device (not illustrated) outside the health management system 3, and the server device can always manage the latest determination target data and position information. In this case, the server device can be configured to return the determination target data together with the position information in a case where a determination target data transmission request has been received from the health management apparatus 10a. As a result, the health management apparatus 10a can use the determination target data as determination target data of a passerby in a predetermined zone indicated by the position information.

Alternatively, the health management apparatus 10a may transmit a determination target data transmission request by broadcasting according to the position information of the wearable device 41 or the terminal apparatus 40, and the wearable device 41 or the terminal apparatus 40 may return the determination target data acquired in advance in response to the transmission. In this case, only in a case where the user of the wearable device 41 or the terminal apparatus 40 permits provision of the information based on the position information, the determination target data may be transmitted when the user enters the predetermined zone.

The health management apparatus 10a can include a second determination unit that determines a health state of a passerby based on the determination target data acquired by the wearable device 41 or the terminal apparatus 40. The second determination unit can also be implemented as a function of the control unit 11. The second determination unit can also determine the health state of each passerby who can acquire the determination target data by using a learning model that receives the determination target data and outputs the health state of each passerby. An algorithm or the like of the learning model is not limited.

Furthermore, the terminal apparatus 40 can also be used to display the index. That is, at least one of the display apparatuses 20 provided in the health management system 3 can be the terminal apparatus 40 used by a passerby. In this case, the control unit 11 can also perform display on the terminal apparatus by specifying a person included in image data by executing one of face authentication processing or gait authentication processing based on the image data, and transmitting a display image to a terminal apparatus associated with the person in advance via the communication unit 13. In this case, information indicating a determination result for the individual and the like may also be displayed together with information of the entire predetermined zone. For example, it is realistic to perform such association only for residents in a predetermined zone, and in this case, it is sufficient if displaying on a visitor's terminal apparatus in the predetermined zone does not be executed.

Alternatively, even in a case where an individual is not specified, the communication unit 13 may be configured to transmit a display image in broadband, and the display image may be displayed in a case where the terminal apparatus 40 has received the display image.

The determination target data thus obtained can be used in calculating the index indicating the group health state as follows.

The learning model 12c stored in the storage unit 12 is a learning model that receives a result of determining movement of a passerby (hereinafter, referred to as first determination result) and a result of determining a health state of the passerby based on determination target data (hereinafter, a second determination result), and outputs, for each predetermined zone, an index indicating a group health state which is a health state of all persons existing in the predetermined zone, and an algorithm or the like thereof is not limited. In addition, the input parameters are not limited thereto.

The learning model 12c can be obtained, for example, as follows. That is, the learning model 12c can be obtained by performing machine learning in a manner in which a data set including the first and second determination results and ground truth data indicating a statistical value (index) obtained by performing statistical processing on the health age or the like of each passerby at this time is input to an untrained learning model. For example, a plurality of data sets in which at least one of a determination timing or the predetermined zone is different from each other can be used for training. The index of the passerby is not limited to such a statistical value, and for example, an index determined by a model creator or the like can be used. The learning model 12c can be a model that can accurately determine the index indicating the group health state by learning the data set for a number of timings or predetermined zones to the extent that over-learning does not occur. Note that a system that performs such learning will be described below.

Then, the control unit 11 can be configured to calculate the index indicating the group health state based on the first determination result and the second determination result by using such a learning model 12c. However, the control unit 11 can also be configured to calculate the index indicating the group health state based on the first determination result and the second determination result without using such a learning model 12c.

As described above, as the health management apparatus 10a is configured to calculate the index by using not only the first determination result but also the second determination result, the health management system 2 can determine a health state of a passerby in a more multidimensional manner, so that information indicating a health state of the entire predetermined zone to be displayed can be made more accurate.

In addition, the health management apparatus 10a may include the resident determination unit that determines whether or not a passerby is a resident residing in a predetermined zone based on image data received from the camera 30, and determines an actual age in a case where the passerby is a resident. The health management apparatus 10a can include, as the resident determination unit, a person information database (DB) 12b, a function of determining whether or not a passerby is a resident while referring to the person information DB 12b, and a function of determining the actual age of the resident, and the control unit 11 can have these functions.

The resident determination unit can determine whether or not a passerby is a resident or determine the actual age of the resident by, for example, at least one of face authentication processing or gait recognition processing. Therefore, the person information DB 12b can be a DB that stores at least one of face image data or gait data and actual age information or personal identification information such as a name or an ID including the actual age information for each user residing in a predetermined zone. In addition, the personal identification information can include information such as an ID for identifying the wearable device 41 or information such as an ID for identifying the terminal apparatus 40 connectable to the wearable device 41 instead of or in addition to the name or ID.

However, the person information DB 12b does not have to include the personal identification information. This is because the control unit 11 can determine whether or not a passerby is a resident by comparing image data with at least one of the face image data or the gait data, and can specify the actual age in a case where the passerby is a resident. The person information DB 12b is a database (DB) that is referred to for determining whether or not a passerby is a resident or the like, and does not need to be stored in a configuration that does not include the resident determination unit.

In a case where the face authentication processing is used for resident determination, the camera 30 may be installed in such a way as to meet conditions such as a position, an imaging direction, and an imaging magnification for imaging the face of the user, to perform imaging. In a case where the gait authentication processing is used for resident determination, the camera 30 does not need to be installed in such a way as to meet such conditions, and it is sufficient that the camera 30 is installed in such a way as to meet conditions such as a position, an imaging direction, and an imaging magnification for imaging a walking figure of the user (that is, imaging the entire user).

For example, the resident determination unit may detect the presence of a person from imaging data obtained by the camera 30 and execute the above-described comparison processing in a case where the person has been detected. That is, the resident determination unit may execute the above-described comparison processing when a person enters an imaging range of the camera 30.

The resident determination unit can also be configured to determine whether or not a passerby is a resident and the actual age of the resident by using a learning model. That is, instead of the person information DB 12b, a learning model that receives at least one of face image data or image data obtained by imaging all residents in a predetermined zone and outputs information indicating the user, the wearable device 41, or the terminal apparatus 40 including the actual age information can be stored in the storage unit 12. At the time of operation, the control unit 11 can input image data captured by the camera 30 to the learning model, obtain information indicating the user, the wearable device 41, or the terminal apparatus 40 including the actual age information, and obtain the actual age information in a case where the user is a resident. In a learning stage, machine learning is executed by a portable terminal apparatus used by each individual, and a learning model as a result of the machine learning, a learned coefficient, or the like is transmitted to the health management apparatus 10a, whereby the health management apparatus 10a can determine whether or not the individual is a resident and determine the actual age in a case where the individual is a resident by using the learning model for each individual.

The control unit 11 can also calculate a first statistical value that is a statistical value of a difference between the health age and the actual age for all the persons existing in the predetermined zone and residing in the predetermined zone based on the result of determining the state of movement of the resident. The health age of each passerby can be estimated based on, for example, a walking speed, a gait (walking pattern), a walking posture, or the like, or based on gray hair, a facial expression (the degree of aging of the face), a stress level, or the like obtained by analyzing the face image data. As the actual age, the actual age information of the passerby determined as a resident can be used. The first statistical value can be obtained by performing statistical processing of the health age with respect to the actual age of each passerby for all residents. Then, control unit 11 calculates a non-resident health age that is a health age for all persons existing in the predetermined zone and not residing in the predetermined zone based on a result of determining states of movement of persons other than the resident. Similarly to the health age of the resident, the non-resident health age can also be estimated from the walking speed, the facial expression, and the like.

Further, the control unit 11 can calculate, as the index or as a part of the index, a statistical value of a difference between the health age and the actual age for all the persons existing in the predetermined zone based on the first statistical value and the non-resident health age. In this manner, the control unit 11 may estimate the statistical value of the difference between the health age and the actual age for all the residents and non-residents with reference to the actual age information for the residents.

The statistical value of the difference between the health age and the actual age can also be calculated using a learning model. For example, by using a supervised learning model that receives a result of determining a state of movement of a resident (the walking state or the health state such as the stress level) and outputs a difference between the health age indicated by a face image or the like and the actual age, a result of determining the state of movement of the residents and non-residents can be input, a difference between the health age and the actual age can be calculated for all passersby, and a statistical value thereof can be calculated. Alternatively, by using a supervised learning model that receives a result of determining of a state of movement of a resident (the walking state, the stress level, or the like) and outputs a statistical value of a difference between the health age indicated by the face image or the like and the actual age, a result of determining states of movement of the residents and non-residents can be input, and a statistical value for all passersby can be calculated. Furthermore, the statistical value of the difference between the health age and the actual age can also be calculated using the learning model 12a by constructing the learning model 12a in such a way that the statistical value becomes a part of output parameters of the learning model 12a.

As the statistical value of the difference between the health age and the actual age is calculated in this manner, the health management system 3 can calculate the difference between the health age and the actual age for all the persons existing in the predetermined zone by using the difference between the health age and the actual age with a certain degree of accuracy for the residents. Furthermore, in the health management system 3 having such a configuration, the persons in the predetermined zone can be made to have a sense of goal based on the calculation result, so that the health level of all the persons existing in the predetermined zone can be further improved.

In addition, the health management system 3 may include an incentive calculation unit that calculates an incentive to be given (granted) for each predetermined zone based on the calculated index. Furthermore, the health management system 3 may include a giving unit that instructs the target management system 21 to give the calculated incentive via the communication unit 13. This incentive calculation unit can be implemented as one function of the control unit 11, and the giving unit (granting unit) can also be implemented as one function of the control unit 11. Management system information 12d stored in the storage unit 12 can be referred to when such an incentive is calculated and the giving instruction is made.

The management system information 12d can include a connection destination such as an internet protocol (IP) address of the target management system 21 and a value to be given in a case of giving the incentive. The management system information 12d can be changed by an administrator or the like. That is, the control unit 11 can include a setting unit (not illustrated) that performs setting for changing the management system information 12d.

For example, in a case where the calculated index such as the health age indicates that the index is higher than a predetermined standard (for example, an average value or a median value for all predetermined zones), the giving unit may provide the incentive to the predetermined zone. The incentive can be given, for example, by giving points or electronic money that can be used at least in a predetermined zone that is a giving target, or by increasing a national supplement in the predetermined zone. In addition, the incentive can be given by lowering an insurance fee for the predetermined zone (discount of the fee) or by increasing funds for health promotion activities in the predetermined zone. In any example, the giving unit may instruct the target management system 21 corresponding to a giving target destination to give the incentive.

With such a configuration, in the health management system 3, since the incentive calculated for each predetermined zone can be given for each predetermined zone, the health level of all the persons existing in the predetermined zone can be further improved.

The health management apparatus 10a may further include a generation unit that generates a change instruction for changing predetermined information regarding life in the predetermined zone based on the index calculated by the function of the calculation unit 1b for each predetermined zone. The life in the predetermined zone can refer to any one or more of food, clothing, and housing in the predetermined zone. Information indicating a change instruction destination and what kind of change is to be made can be stored in the storage unit 12, for example.

The change instruction destination can be, for example, a predetermined facility, a store, a fare management system, or the like, and in this case, the change instruction can be an instruction to discount or increase a fare related to food, clothing, and housing as a whole, such as a usage fee of a public facility, a taxi, or a bus, or a meal fee in a restaurant. For example, in a case where the health level is lower than a predetermined value or lower than the past by a predetermined value, the control unit 11 can notify a certain store of an instruction to increase the price of ramen by a certain amount and to decrease the price of soba by a certain amount, and can cause a display apparatus 21a of the store to display a menu image reflecting the instruction as illustrated in FIG. 8.

Furthermore, the change instruction destination may be, for example, a predetermined moving apparatus (an elevator, an escalator (stepwise lifting device), a horizontal escalator, or the like, and the change instruction may be an instruction to change a moving speed of the moving apparatus. For example, in a case where the health level is lower than a predetermined value or lower than the past by a predetermined value, the control unit 11 can decrease the moving speed of the moving apparatus, so that people do not get on the moving apparatus and do not have the feeling of wanting to get on the moving apparatus. In this way, it is possible to induce an increase in exercise load by performing control to increase inconvenience.

By including such a generation unit, in the health management system 3, predetermined information regarding life in the predetermined zone can be changed, and people can act based on the changed information, so that the health level of all the persons existing in the predetermined zone can be further improved.

The generation unit may generate the change instruction for each attribute of a resident residing in the predetermined zone. The resident referred to herein is not basically limited to a resident captured as a passerby by the camera 30, and the change instruction can be generated for stores and facilities used by general residents. Furthermore, the attribute can be, for example, an attribute distinguished by a healthy person, a wand, an elderly person, a disabled person, or the like, an attribute distinguished by sex, age, walking ability, or the like of a passerby, or the like. The attribute can be determined from image data acquired by the camera 30, and can also be determined from personal identification information obtained by communication with the terminal apparatus 40 or the like. Furthermore, the control unit 11 can also display a detour route only for a resident with a certain attribute at the time of setting a navigation route of the terminal apparatus 40 according to the calculated index.

By including such a generation unit, in the health management system 3, predetermined information regarding life in the predetermined zone can be changed for each resident attribute, and residents can act based on the changed information, so that the health level of all the persons existing in the predetermined zone can be further improved.

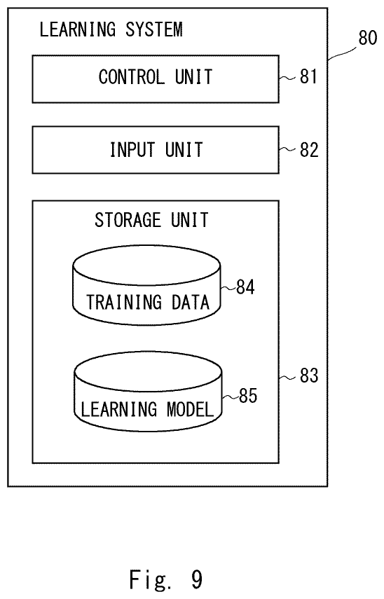

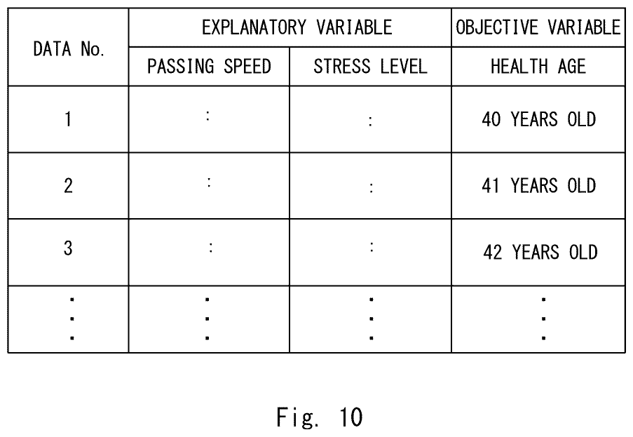

Finally, a configuration example of a learning system that generates the above-described various learning models will be described with reference to FIGS. 9 and 10. FIG. 9 is a block diagram illustrating a configuration example of the learning system that generates the learning model used in the health management system 2 of FIG. 3 or the health management system 3 of FIG. 7. FIG. 10 is a diagram illustrating an example of training data used in the learning system of FIG. 9.

A learning system 80 illustrated in FIG. 9 can include a control unit 81, an input unit 82, and a storage unit 83. The learning system 80 can be constructed using, for example, a computer such as an artificial intelligence (AI) training PC. However, the learning system 80 may be implemented by a single apparatus or may be implemented by distributing functions to a plurality of apparatuses.

The control unit 81 controls the entire learning system 80. The control unit 81 can be implemented by, for example, an integrated circuit, and can be implemented by, for example, a processor, a work memory, a nonvolatile storage apparatus, and the like. A control program executed by the processor is stored in the storage apparatus, and the processor reads the program to the work memory and executes the program, so that the function of the control unit 81 can be performed. The control program can include a training program for executing training. As the storage apparatus, the storage unit 83 can also be used.

The input unit 82 can include at least one of an interface for performing a data input operation or a communication interface for inputting data from an external apparatus by communication. The input unit 82 inputs a data set of the training data 84 necessary for training, and stores the data set in the storage unit 83 so that the data set can be referred to during training. The storage unit 83 can store the training data 84 and can store a learning model 85 as an untrained model.

In the processing performed by the learning system 80, it is sufficient if the control unit 81 causes the learning model 85 as an untrained model to perform machine learning based on the training data 84, thereby training the learning model 85 to be a trained model. Furthermore, in a case where retraining is necessary, the learning model 85 as a trained model can be retrained based on a newly prepared data set.

For example, as described above, the training data 84 in a case of generating the learning model 12a can be a data set including the result of determining movement of each passerby and the ground truth data indicating the statistical value (index) obtained by performing the statistical processing on the health age or the like of each passerby at this time. For example, a plurality of data sets in which at least one of a determination timing or the predetermined zone is different from each other can be used for training.

In addition, as described above, for example, the training data 84 in a case of generating the learning model 12c can be a data set including the first determination result, the second determination result, and the ground truth data indicating the statistical value (index) obtained by performing the statistical processing on the health age or the like of each passerby at this time. More specifically, the training data 84 in a case of generating the learning model 12c can be a data set as illustrated in FIG. 10. The data set illustrated in FIG. 10 includes the passing speed of each passerby obtained as the first determination result and the stress level of each passerby obtained as the second determination result as explanatory variables, and includes the health ages of all the persons existing in the predetermined zone as an objective variable. Although only the passing speed and the stress level of one passerby are illustrated as the explanatory variables in FIG. 10 for convenience, the passing speeds and the stress levels of a maximum number of passersby may be used as the explanatory variables.

In addition, in a case where the control unit 11 calculates the index by using a learning model that outputs the index from image data obtained by imaging a passerby with the camera 30 installed in a predetermined zone, the training data 84 in a case of generating the learning model can be, for example, the image data acquired by the camera 30 (or a pixel group constituting the image data) as the explanatory variable and the health ages of all the persons existing in the predetermined zone as the objective variables. Even in a case where another learning model is generated, a similar learning system can be used only with a difference in the algorithm, the training data, and the like.

### Other Embodiments

The health management system according to the embodiment described above is not limited to the configuration example described above and is not limited to the configuration for executing the processing example described above as long as the functions may be implemented.

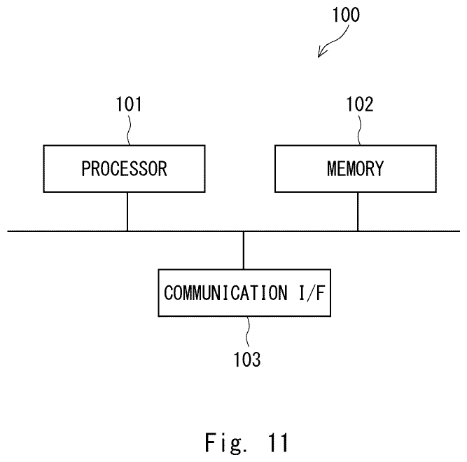

In addition, each apparatus included in the health management system described in the above embodiment can have the following hardware configuration. FIG. 11 is a diagram illustrating an example of a hardware configuration included in an apparatus.

An apparatus 100 illustrated in FIG. 11 is each apparatus in the health management system according to the above embodiment, and includes a processor 101, a memory 102, and a communication interface (I/F) 103. The processor 101 may be, for example, a CPU, a graphics processing unit (GPU), a micro processor unit (MPU) which is also referred to as a microprocessor, or the like. The processor 101 may include a plurality of processors.

The function of each unit in each apparatus can be implemented by the processor 101 reading a program stored in the memory 102 and executing the program in cooperation with the communication I/F 103. Note that at least one of a wireless communication I/F or a wired communication I/F is provided in the communication I/F 103 in some apparatuses. In addition, each apparatus can include, for example, an I/F for a sensor, an input/output apparatus, or the like necessary for the apparatus.

The program includes instructions (or software codes) that, when loaded into a computer, cause the computer to perform one or more of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

According to the present disclosure, it is possible to provide the health management system, the health management method, and the program capable of enhancing health awareness of persons existing in a predetermined zone and improving a health level of all the persons existing in the predetermined zone.

Note that the present disclosure is not limited to the above embodiment, and can be appropriately changed without departing from the scope. In addition, the present disclosure includes an appropriate combination of the examples in the above embodiment.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A health management system executing:
first determination processing of determining a state of movement of a passerby based on image data obtained by imaging the passerby with a camera installed in a predetermined zone;
calculation processing of calculating an index indicating a group health state which is a health state of all persons existing in the predetermined zone based on a determination result in the first determination processing; and
display processing of displaying the index calculated in the calculation processing.

2. The health management system according to claim 1, wherein the display processing includes processing of displaying the index calculated for each predetermined zone in a ranking format.

3. The health management system according to claim 1, wherein the display processing includes processing of displaying the index on a display apparatus disposed outdoors in the predetermined zone.

4. The health management system according to claim 1, wherein the first determination processing includes processing of determining the state of movement of the passerby, and the first determination processing includes determining a health state of the passerby based on face image data included in the image data.

5. The health management system according to claim 1, wherein
the calculation processing includes processing of calculating a statistical value of passing speeds of all the persons existing in the predetermined zone based on the determination result in the first determination processing, and
the display processing includes processing of displaying the statistical value as the index or as a part of the index.

6. The health management system according to claim 5, wherein
the calculation processing includes processing of determining a target value of the passing speed based on the statistical value, and
the display processing includes processing of displaying the statistical value and the target value as the index or as a part of the index.

7. The health management system according to claim 1, further executing resident determination processing of determining whether or not the passerby is a resident residing in the predetermined zone based on the image data, and determining an actual age of the resident in a case where the passerby is the resident,
wherein the calculation processing includes processing of calculating a first statistical value that is a statistical value of a difference between a health age and an actual age for all persons existing in the predetermined zone and residing in the predetermined zone based on the determination result in the first determination processing for the resident, calculating a non-resident health age that is a health age for all persons existing in the predetermined zone and not residing in the predetermined zone based on the determination result in the first determination processing for persons other than the resident, and calculating a statistical value of a difference between a health age and an actual age for all the persons existing in the predetermined zone based on the first statistical value and the non-resident health age as the index or as a part of the index.

8. The health management system according to claim 1, further executing:
acquisition processing of acquiring determination target data used for determining the health state of the passerby from a terminal apparatus carried by the passerby; and
second determination processing of determining the health state of the passerby based on the determination target data acquired in the acquisition processing,
wherein the calculation processing includes processing of calculating the index based on the determination result in the first determination processing and a determination result in the second determination processing.

9. The health management system according to claim 1, further executing generation processing of generating a change instruction for changing predetermined information regarding life in the predetermined zone based on the index calculated in the calculation processing for each predetermined zone.

10. The health management system according to claim 9, wherein the generation processing includes processing of generating the change instruction for each attribute of a resident residing in the predetermined zone.

11. The health management system according to claim 1, further executing incentive calculation processing of calculating an incentive to be given for each predetermined zone based on the index calculated in the calculation processing.

12. A health management method comprising:
determining, by a computer, a state of movement of a passerby based on image data obtained by imaging the passerby with a camera installed in a predetermined zone;
calculating, by the computer, an index indicating a group health state which is a health state of all persons existing in the predetermined zone based on a result of the determination; and
performing, by the computer, control to display the calculated index on a display apparatus externally connected to the computer or included in the computer.

13. A non-transitory computer readable medium storing a program for causing a computer to execute processing of:
determining a state of movement of a passerby based on image data obtained by imaging the passerby with a camera installed in a predetermined zone;
calculating an index indicating a group health state which is a health state of all persons existing in the predetermined zone based on a result of the determination; and
displaying the calculated index on a display apparatus externally connected to the computer or included in the computer.
